(19) United States
(12) Patent Application Publication
(10) Pub. No.: US 2024/0199754 A1
(43) Pub. Date: Jun. 20, 2024

(54) Applications of GHR-106 Monoclonal Antibody as a GnRH Antagonist

(71) Applicant: VANCOUVER BIOTECH LTD., Vancouver (CA)

(72) Inventor: Chi-Yu Gregory Lee, Vancouver (CA)

(21) Appl. No.: 18/286,895

(22) PCT Filed: May 17, 2022
(86) PCT No.: PCT/CA2022/050777

§ 371 Date: Oct. 13, 2023

Related U.S. Application Data
(60) Provisional application No. 63/189,852, filed on May 18, 2021.
(60) Provisional application No. 63/242,976, filed on Sep. 10, 2021.

(51) Int. Cl. C07K 16/28; A61K 39/00; A61P 5/24

(52) U.S. Cl. CPC C07K 16/2869; A61P 5/24; A61K 2039/505; A61K 2039/545; C07K 2317/24; C07K 2317/33; C07K 2317/52; C07K 2317/565; C07K 2317/76

(57) ABSTRACT

GHR-106 monoclonal antibody or antigen-binding fragments thereof are provided and used to modulate levels of reproductive hormones in vivo when administered to mammalian subjects. The GHR-106 monoclonal antibody or an antigen-binding fragment thereof can be used to control ovulation, terminate ectopic pregnancy, and/or treat reproductive disorders or conditions in mammalian subjects.

### CROSS-REFERENCE TO RELATED APPLICATIONS

This application claims priority to, and the benefit of, U.S. provisional patent application No. 63/189,852 filed 18 May 2021 and No. 63/242,976 filed 10 Sep. 2021, both entitled Applications of GHR-106 Monoclonal Antibody as GnRH Antagonist. Both of the foregoing applications are hereby incorporated by reference in their entireties for all purposes.

### TECHNICAL FIELD

Some embodiments relate to compositions for treating reproductive disorders in mammalian subjects. Some embodiments relate to compositions for modulating a level of reproductive hormones in mammalian subjects.

### BACKGROUND

GnRH (gonadotropin releasing hormone) is a decapeptide hormone which reacts with the GnRH receptor located in the human anterior pituitary to control the release or secretion of luteinizing hormone (LH) and follicle stimulating hormone (FSH). These two reproductive hormones are essential for sexual differentiation and maturation of reproductive systems in all animal species including humans.

GHR-106 is a monoclonal antibody generated from mouse against the N1-29 oligopeptide located in the extracellular domains of human GnRH (Gonadotropin releasing hormone) receptor. As can be seen from FIG. 1A, which shows comparisons of the N1-29 oligopeptides located in the extracellular domains of the GnRH receptor from several species (SEQ ID NO:1 to SEQ ID NO:6), there is a high degree of amino acid sequence homology (between about 90-95%) between human, rabbit, monkey, cat and dog, and a lesser degree of sequence homology with mouse.

FIG. 1B shows the amino acid sequences of the heavy chain and light chain of the IgG4 humanized GHR-106 monoclonal antibody (SEQ ID NO:7 and SEQ ID NO:8, respectively) and further identifies by underlining the complementarity determining regions CDR1, CDR2 and CDR3 of both the heavy chain (SEQ ID NO:9 to SEQ ID NO:11) and the light chain (SEQ ID NO: 12 to SEQ ID NO: 14).

The illustrated embodiment of the humanized IgG4 GHR-106 contains a S228P mutation engineered into the heavy chain of the antibody, as seen in SEQ ID NO:7 (note that S228 according to the EU numbering system is at position 250 in the amino acid SEQ ID NO:7). Without being bound by theory, it is believed that the S228P mutation or other equivalent mutation prevents the antibody from undergoing a recombinant process known as IgG4 Fab-arm exchange. Fab-arm exchange results in the formation of unwanted bispecific antibodies, which is known to have an undesirable effect on the specificity of the antibody to the target receptor. See, for example, Silva et al., JBC, 2015, 290(9):5462-5469, which is incorporated by reference herein for all purposes.

Due to high degrees of amino acid sequence homology (>90-95%), human GHR-106 cross-reacts with the N1-29 peptides of monkey, rabbit, dog or cat GnRH, but not with those of mouse and rat. GHR-106 and its humanized forms have been shown to react specifically with human GnRH receptor either in cancer cells or in the anterior pituitary.

In humans, there is only one type of functional GnRH receptor gene. The main site of action of the GnRH receptor located in the anterior pituitary is responsible for the release of gonadotropin hormones, luteinizing hormone (LH) and follicle stimulating hormone (FSH), upon pulsatile stimulation of GnRH released from hypothalamus. However, in the reproductive related tissues or organs such as ovary or testis, as well as cancer cells, the presence of the GnRH receptor can serve to react with GnRH or its peptide analogs upon binding interactions through the mechanism of autocrine/paracrine regulation.

The administration of GnRH analogs that are antagonistic to the normal function of GnRH has been used for the treatment of a variety of sex hormone-related conditions or disorders such as reproductive diseases (in both males and females), infertility, assisted reproductive therapy such as in vitro fertilization (IVF) or egg donation (e.g. to control ovarian stimulation), contraception including inhibition of ovulation, medical transition for transgender people or sex reassignment therapy, whether male-to-female or female-to-male, and whether in conjunction with sex reassignment surgery or not, endometriosis, endometrial thinning, adenomyosis, endometrial hyperplasia, uterine leiomyoma (uterine fibroids), premenstrual syndrome, benign prostatic hypertropy, ovarian disorders, polycystic ovary disease, precocious puberty, and the like.

Due to the relatively short half-life of the native form of the hormone (2-4 min), numerous decapeptides and derivatives were made with the objective of increasing their circulation half-life to hours. Due to various structural modifications, some retain similar biological actions to stimulate or inhibit the release of gonadotropins and they are generally termed as GnRH agonists or GnRH antagonists, respectively, with respect to their mechanism of biological action to stimulate or inhibit the release of gonadotropin hormones. Decades ago, cetrorelix was released in the market and served as a GnRH antagonist for application as drugs for fertility regulation or as anti-cancer drugs with higher potency and longer half-life (hours vs. minutes) than the native GnRH. Examples of synthetic GnRH antagonists include, among others, antide, cetrorelix, abarelix, degarelix, ganirelix and elagolix.

Previous work (see U.S. Pat. Nos. 8,163,283, 9,273,138, and publication No. 2020/035462, each of which is incorporated by reference herein) related to potential clinical applications of GHR-106 and its humanized forms in treatment of human cancer and possibly fertility-related diseases. PCT application publication No. WO 2019/153075, which is incorporated by reference herein, discloses that the monoclonal antibody GHR-106 acts against human GnRH receptor and can be potentially developed into a long-acting GnRH antagonist but is otherwise biosimilar to Cetrorelix or other established peptide analogues. This is due to the fact that antibody drugs generally have a much longer half-life of 5-21 days as compared to hours for peptide antagonists such as the known GnRH peptide antagonists Cetrorelix or Antide. Despite the difference in molecular size (80 kDa vs. ˜1.5 kDa), both the peptides and the GHR-106 antibody were demonstrated to exhibit similar binding affinity (K_{d} 1-4 nM) and specificity to human GnRH receptor. GHR-106 monoclonal antibody has a half-life of 5-21 days, whereas the half-life of peptide GnRH antagonists such as Cetrorelix or Antide is 1-10 hrs in most cases.

Numerous GnRH peptide analogs or derivatives are currently available for clinical applications as drugs in cancer treatments such as prostate and breast cancer. Clinical applications also include many indications related to women's health, fertility and disease conditions. For example, GnRH peptide analogs are widely used for in vitro fertilization (IVF) to control programmed ovulation and hormone-dependent diseases such as endometriosis, uterine fibroids and premenstrual syndrome, or polycystic ovarian syndrome.

There is a general desire for improved and/or longer acting compositions that can be used to treat reproductive disorders and/or modulate the level of reproductive hormones in a mammalian subject.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

GHR-106 monoclonal antibody or an antigen-binding fragment thereof can be used to regulate a level of a sex related hormone in a mammalian subject. The GHR-106 monoclonal antibody or antigen-binding fragment thereof can cause reversible suppression of at least one sex related hormone in the mammalian subject. The reversible suppression of the at least one sex related hormone can be a decrease in serum levels of the at least one sex related hormone in the subject for a period of between 3 days and 21 days after administration of the GHR-106 monoclonal antibody or antigen-binding fragment thereof to the subject. The at least one sex related hormone can be testosterone, estradiol, luteinizing hormone, progesterone, follicle stimulating hormone, or a combination thereof. The GHR-106 monoclonal antibody or antigen-binding fragment thereof can be adapted for administration at a dosage of between about 1 mg/kg to about 3 mg/kg relative to the weight of the subject. For humans, this can translate to a dose of about 50 mg to about 300 mg. the administration can be repeated at regular spaced apart intervals, for example between about every 1 week to about every 3 weeks.

In some aspects, the GHR-106 monoclonal antibody or antigen-binding fragment thereof can have a heavy chain having CDRs having each of SEQ ID NO:9, SEQ ID NO: 10 and SEQ ID NO: 11, and a light chain having CDRs having each of SEQ ID NO: 12, SEQ ID NO: 13 and SEQ ID NO: 14.

In some aspects, the GHR-106 antibody or antigen-binding fragment thereof can be used to terminate an ectopic pregnancy, can be used to control ovulation, can be used for fertility control in a male or female subject, and/or can be used in the treatment of a sex hormone-related condition or disorder. The subject can be a mammalian subject, including a human, monkey, dog, cat, rabbit, or the like. Methods embodying any of the foregoing uses are also provided.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1A shows comparisons of the amino acid sequences corresponding to N1-29 oligopeptides located in the extracellular domains of GnRH receptor from humans, rabbit, monkey, cat, dog and mouse.

FIG. 1B shows the amino acid sequences of the heavy chain and light chain of the GHR-106 antibody with complementarity determining regions (CDRs) underlined.

FIG. 2 shows a double log plot for ΔOD at 405 nm vs. GHR-106 antibody concentration GHR-106 monoclonal antibody applied to three separate well-coated N1-29 synthetic oligopeptides of GnRH receptor from humans, dog and rabbit, respectively.

FIG. 3 shows serum LH (mIU/mL) and testosterone (ng/ml) concentrations plotted as a function of days after a single injection with 3 mg/kg of GHR-106 subcutaneously on day 1 to an adult male rabbit. The hormone levels were monitored from day −1 to day 30.

FIG. 4 shows serum LH (mIU/mL) and estradiol (E2, pg/mL) concentrations plotted as a function of days after a single subcutaneous injection with 3 mg/kg of GHR-106 on day 1 to an adult female rabbit. The hormone levels were monitored from day 1 to day 20.

FIG. 5 shows quantitative RT-PCR with gene expression levels of OC-3-VGH ovarian cancer cells to reveal changes in gene regulation upon treatments of cancer cells with Antide (peptide antagonist) and GHR-106, respectively.

FIG. 6 shows serum testosterone levels of a total ten male rabbits divided into three experimental groups.

FIG. 7 shows serum LH levels from a total of ten male rabbits divided into three experimental groups.

FIG. 8 shows serum estradiol (E2) levels for a total of ten female rabbits divided into three experimental groups, which were maintained from day 1 to day 17.

FIG. 9 shows serum LH levels from a total of ten female rabbits divided into three experimental groups during the study period.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

The inventor has now carried out investigations including proof-of-concept experiments in rabbits and quantitative gene regulation studies as disclosed herein to support the wide-spread clinical application of GHR-106 for treatment of fertility problems and other reproductive disorders in several animal species, including humans. The proof-of-concept experiments described herein conducted in rabbits revealed reversible suppressions of serum reproductive hormones (LH, testosterone or estradiol) over a period of approximately one or two weeks. This new data demonstrates the potential suitability of GHR-106 for therapeutic application in humans as well as several other animal species.

It has not previously been demonstrated that GHR-106 will also act on the GnRH receptor in the anterior pituitary in vivo in a manner similar to decapeptide GnRH antagonists, which are known to suppress the release of gonadotropins. Therefore, in this study, the rabbit was selected as a proof-of-concept animal model to demonstrate that GHR-106 acts on the pituitary GnRH receptor to suppress the release of gonadotropin hormones in vivo. Therefore, through comparisons of bio-similarity in terms of the effect on gene expression and the regulation of the levels of reproductive hormones in vivo, it is reasonable to assume that GHR-106 could serve as an alternative to the known peptide GnRH antagonists for the therapeutic treatments of many gynecological diseases or reproductive disorders, besides human cancer, but with potential benefits associated with its longer half-life.

In some embodiments, a GHR-106 antibody or an antigen-binding fragment thereof is provided. In some embodiments, the GHR-106 antibody or antigen-binding fragment thereof is administered to a mammal, including for example to a human, monkey, dog, cat, horse, cow, sheep, goat, rabbit or other domestic animal, to treat a reproductive condition or disorder or a sex hormone-related health condition. In some embodiments, the GHR-106 antibody or antigen-binding fragment thereof is administered to a mammal in which the N1-29 terminal amino acid sequence of the GnRH receptor has an amino acid sequence with at least 90% sequence identity to the human N1-29 terminal amino acid sequence of the GnRH receptor (SEQ ID NO:1), including e.g. at least 91%, 92%, 93%, 94%, 95%, 96%, 97%, 98% or 99% sequence identity thereto.

In some embodiments, the GHR-106 antibody or antigen-binding fragment thereof is a chimeric antibody that is engineered to minimize the likelihood of cross-reactivity of the antibody in the target species. For example, the GHR-106 IgG4 construct disclosed herein having a heavy chain with the amino acid sequence of SEQ ID NO:7 and a light chain with the amino acid sequence of SEQ ID NO:8 is a humanized antibody construct. In other embodiments in which the subject is a different mammalian species, chimeric antibodies engineered to contain the Fc regions of IgG4 from the subject's species could be used, for example dog IgG4-Fc for an antibody intended for administration to dogs, cat IgG4-Fc for an antibody intended for administration to cats, rabbit IgG4-Fc for an antibody intended for administration to rabbits, monkey IgG4-Fc for an antibody intended for administration to monkeys, horse IgG4-Fc for an antibody intended for administration to horses, bovine IgG4-Fc for an antibody intended for administration to cows, sheep IgG4-Fc for an antibody intended for administration to sheep, goat IgG4-Fc for an antibody intended for administration to goats, and so on.

In some embodiments, the GHR-106 antibody is provided as one or more active antigen-binding fragments of GHR-106 for use in treating sex hormone-related health conditions or disorders. In some embodiments, the fragments are single chain fragments of the variable regions of GHR-106. In some embodiments, the fragments are fragments of GHR-106 of the IgG isotype, including IgG4. In some embodiments, the fragment is an F(ab′)2 fragment. In some embodiments, the F(ab′)2 fragment has a molecular weight of 110 KDa. In some embodiments, the fragment is a Fab fragment. In some embodiments, the Fab fragment has a molecular weight of 55 KDa. In some embodiments, the fragment is an scFab fragment. In some embodiments, the scFab fragment has a molecular weight of 25 KDa. In some embodiments, the fragment is an scFv fragment. In some embodiments, the scFv fragment has a molecular weight of 25 KDa. In some embodiments, combinations of different antigen-binding fragments e.g. two or more of the fragments as described above, can be used as a drug for the treatment of a sex-hormone related condition or disorder.

In some embodiments, the GHR-106 antibodies or antigen-binding fragments thereof that are administered for the treatment of sex hormone-related health conditions or disorders do not possess effector functions. An antibody that does not possess effector functions cannot activate, for example, complement-dependent cytoxicity (CDC) or antibody-dependent cellular cytotoxicity (ADCC) pathways. In some embodiments, the GHR-106 antibodies or antigen-binding fragments thereof that do not possess effector functions have an IgG4 subtype. In some embodiments, the GHR-106 antibodies or antigen-binding fragments thereof inhibit complement activation. In some embodiments, the heavy chain of the antibody having the IgG4 subtype has a S228P mutation or an equivalent mutation, to prevent Fab-arm exchange. In some embodiments, the GHR-106 antibodies or antigen-binding fragments thereof that do not possess effector functions are IgG antigen-binding fragments of GHR-106 antibodies. In some embodiments, the antigen-binding fragments that do not possess effector functions are F(ab′)₂, Fab, scFab or scFv IgG fragments of GHR-106 antibodies. In some embodiments, the GHR-106 antibodies or antigen-binding fragments thereof are derived from hGHR-106.

In some embodiments, the subtype of the GHR-106 antibody is selected to modulate the effector functions of the antibody. In some embodiments, the GHR-106 antibody or antigen-binding fragment thereof is structurally modified to further modulate the effector functions of the antibody, for example by using an antigen-binding fragment of the antibody that does not possess any effector functions. In some embodiments, the Fc region of the GHR-106 antibody is of the IgG4 subtype. In some embodiments, the GHR-106 antibody or antigen-binding fragment thereof that does not possess any effector functions is used for the treatment of a sex hormone-related health condition or disorder, for reversibly suppressing a level of at least one sex related hormone in a subject, for controlling ovulation in a subject, and/or for terminating an ectopic pregnancy in a subject. In some embodiments, the GHR-106 antibody having an IgG4 subtype is used for the treatment of sex hormone-related health conditions or disorders.

Without being bound by theory, it is believed that because the IgG4 antibody subtype does not activate complement-dependent cytotoxicity (CDC) or antibody-dependent cellular cytotoxicity (ADCC), use of the IgG4 antibody subtype for treatment of sex hormone-related health conditions or disorders or otherwise to modulate a level of a sex-related hormone in a subject, including treating fertility disorders, will minimize or eliminate the possibility of CDC and ADCC reactions upon the GHR-106 antibody binding to the anterior pituitary. See for example Vidarsson et al., Front. Immunol., 2014, 5:520, which is incorporated by reference herein for all purposes.

Further, it has been demonstrated that IgG4 antibodies can actually inhibit complement activation (see e.g. van der Zee et al., Clin. Exp. Immunol., 1986, 64(2):415-422, which is incorporated by reference herein for all purposes). Thus, in some embodiments, the GHR-106 monoclonal antibody or antigen-binding fragment thereof is selected to inhibit complement activation. In some embodiments, the GHR-106 monoclonal antibody or antigen-binding fragment thereof that inhibits complement activation is used to treat a sex hormone-related condition or disorder.

In some embodiments, the circulation half-life of the GHR-106 antibody is approximately 3 to 21 days, including any value therebetween e.g. 4, 5, 6, 7, 8, 9, 10, 11, 12, 13, 14, 15, 17, 17, 18, 19 or 20 days, or 72 to 500 hours, including any value therebetween, e.g. 75, 100, 125, 150, 175, 200, 225, 250, 275, 300, 325, 350, 375, 400, 425, 450, or 475 hours. By contrast, the circulation half life cetrorelix is in a range of approximately 10 to 63 hours. GHR-106 has a much longer half life compared to the decapeptide GnRH antagonist cetrorelix, and therefore may require less frequent administration, which may improve patient compliance and/or the feasibility of a proposed treatment regime.

In some embodiments, the IgG antigen-binding fragments that are derived from GHR-106, e.g. F(ab′)2, Fab, ScFab or ScFv, each has a circulation half-life of approximately 12 to 20 hours, including any value therebetween e.g. 13, 14, 15, 16, 17, 18 or 19 hours. The antigen-binding fragments of mGHR-106 or hGHR-106 have a shorter half-life compared to the mGHR-106 or hGHR-106 antibodies. In some embodiments, protein engineering is used to provide GHR-106 antibodies or antigen-binding fragments thereof that have a half-life within a desired range.

In some embodiments, the GHR-106 antibody or the antigen-binding fragment thereof has a heavy chain with the amino acid sequence according to SEQ ID NO:7 and a light chain with the amino acid sequence according to SEQ ID NO:8. In some embodiments, the GHR-106 antibody or antigen-binding fragment thereof has a heavy chain having an amino acid sequence with at least 90% sequence identity to SEQ ID NO:7 and a light chain having an amino acid sequence with at least 90% sequence identity to SEQ ID NO:8, including e.g. at least 91%, 92%, 93%, 94%, 95%, 96%, 97%, 98% or 99% sequence identity to SEQ ID NOS:7 and 8, respectively.

In further embodiments, the GHR-106 antibody or antigen-binding fragment thereof has a heavy chain having the following complementarity determining regions (CDRs): a CDR1 region having the amino acid sequence according to SEQ ID NO:9 (RYSVH), a CDR2 region having the amino acid sequence according to SEQ ID NO: 10 (MIWGGGSTDYNPSLKSR), and a CDR3 region having the amino acid sequence according to SEQ ID NO:11 (GYYSFA). In further embodiments, the GHR-106 antibody or antigen-binding fragment thereof has a light chain having the following CDRs: a CDR1 region having the amino acid sequence according to SEQ ID NO: 12 (KSSQSLLNSRTRKNYLA), a CDR2 region having the amino acid sequence according to SEQ ID NO: 13 (WASTRES), and a CDR3 region having the amino acid sequence according to SEQ ID NO: 14 (KQSYNLYT).

The GHR-106 antibodies or antigen-binding fragments thereof described herein can be formulated in any suitable manner for administration as a medicament. Thus, they can be combined with pharmaceutically acceptable excipients or other pharmaceutically suitable compounds to provide pharmaceutical compositions useful for the modulation of levels of sex hormones and/or treatment of sex hormone-related health conditions or disorders.

In some embodiments, the GHR-106 antibodies or antigen-binding fragments thereof are administered in a therapeutically effective amount for the treatment of sex hormone-related health conditions or disorders and/or in an amount effective to modulate a level of one or more sex related hormones in a mammal, including a human, monkey, dog, cat, rabbit, horse, cow, sheep, goat or other domestic animal. The mammal may be a male or a female. In some embodiments, the sex hormone-related health condition or disorder is a reproductive disease (in a male or female subject), medical transition for transgender people including male-to-female (MTF) or female-to-male (FTM) sex reassignment therapy, whether or not accompanied by sex reassignment surgery, in vitro fertilization (IVF) or egg donation (e.g. to control ovarian stimulation), contraception including inhibition of ovulation in female subjects or of sperm production in male subjects, endometriosis, endometrial thinning, adenomyosis, endometrial hyperplasia, uterine leiomyoma (uterine fibroids), premenstrual syndrome, benign prostatic hypertrophy, ovarian disorders, polycystic ovary disease, precocious puberty, and the like.

In some embodiments, the GHR-106 antibodies or antigen-binding fragments thereof are administered to a male subject for fertility control (e.g. birth control). Without being bound by theory, the data contained in the examples of this application support that administration of GHR-106 antibodies or antigen-binding fragments thereof to a male subject can decrease levels of sex related hormones such as testosterone to a level that is likely to interfere with the production of sperm by the male subject, thereby providing fertility control for a male subject (i.e. birth control for a male subject).

In some embodiments, the GHR-106 antibodies or antigen-binding fragments thereof are administered to terminate an ectopic pregnancy. Without being bound by theory, it is believed that the drop in reproductive hormone levels caused by the administration of a GHR-106 antibody or antigen-binding fragment thereof will be deleterious to the fetus, and/or that GnRH receptor and GnRH are highly expressed in human placenta in parallel with the amount of human chorionic gonadotropin secretion, resulting in the rapid termination of the ectopic pregnancy while minimizing negative effects on the subject.

In some embodiments, the GHR-106 antibodies or antigen-binding fragments thereof are used to regulate ovulation in a subject. In some embodiments, the GHR-106 antibodies or antigen-binding fragments thereof are administered to a female subject for fertility control (e.g. birth control). In some embodiments, the GHR-106 antibodies or antigen-binding fragments thereof are used to regulate a level of one or more sex related hormones in a subject, including by causing a reversible decrease in a serum concentration of the one or more sex related hormones. In some embodiments, the sex related hormone is testosterone, estradiol, lutenizing hormone, progesterone, follicle stimulating hormone, or a combination thereof. In some embodiments, alteration of the level of the sex related hormone alters the fertility status of the subject.

In some embodiments, the GHR-106 antibodies or antigen-binding fragments thereof act as GnRH antagonists in the treatment of any condition that can be treated by known GnRH antagonists including antide or cetrorelix. In some embodiments, the GHR-106 antibodies or antigen-binding fragments thereof are used in the treatment of a condition in which a longer half-life than that of known GnRH antagonists, including antide or cetrorelix, is desirable.

In some embodiments, the GHR-106 antibodies or antigen-binding fragments thereof are administered to a subject at dosage levels of 0.5-10 mg/kg, including any value therebetween, including e.g. about 1 to about 3 mg/kg in some embodiments, e.g. 1.0, 1.5, 2.0, 2.5, 3.0, 3.5, 4.0, 4.5, 5.0, 5.5, 6.0, 6.5, 7.0, 7.5, 8.0, 8.5, 9.0, or 9.5 mg/kg including any value or subrange therebetween. In some embodiments in which the binding affinity and/or specificity of the GHR-106 antibody or antigen-binding fragment thereof has been modified, the dosage level of the modified antibody or antigen-binding fragment thereof is modified appropriately.

In some embodiments in which the subject is a human, the GHR-106 antibody or antigen-binding fragment thereof is administered at a dose of between about 50 mg and about 300 mg, including any value therebetween, e.g. 75, 100, 125, 150, 175, 200, 225, 250 or 275 mg.

In some embodiments, the GHR-106 antibodies or antigen-binding fragments thereof are administered at repeated spaced apart intervals, for example every 5-30 days or any value therebetween, e.g. every 6, 7, 8, 9, 10, 11, 12, 13, 14, 15, 16, 17, 18, 19, 20, 21, 22, 23, 24, 25, 26, 27, 28 or 29 days; every 1-8 weeks or any value therebetween, e.g. every 2, 3, 4, 5, 6 or 7 weeks, or every 2-6 months or any value therebetween, e.g. every 3, 4 or 5 months. In some embodiments, the GHR-106 antibody or antigen-binding fragment there of is administered at repeated spaced apart intervals of between about every 1 week to about every 3 weeks. In some embodiments, the GHR-106 antibodies or antigen-binding fragments thereof that are administered to a human are humanized GHR-106 antibodies or antigen-binding fragments thereof. In some embodiments, the humanized GHR-106 antibody is hGHR-106 IgG4 having a heavy chain having the amino acid sequence of SEQ ID NO:7 and a light chain having the amino acid sequence of SEQ ID NO:8.

A typical route of administration of pharmaceutical compositions comprising antibodies is via injection, typically intravenous or intramuscular. However, any suitable mode of administration can be used in various embodiments.

### EXAMPLES

Certain embodiments are further described with reference to the following examples, which are intended to be illustrative and not limiting in nature.

Proof-of-concept experiments were performed in rabbits to demonstrate reversible suppression of serum reproductive hormones upon a single injection of humanized monoclonal antibody against GnRH receptor, GHR-106(hIgG4) having a heavy chain having the amino acid sequence of SEQ ID NO:7 and a light chain having the amino acid sequence of SEQ ID NO:8.

A single subcutaneous injection of 1 mg/kg or 3 mg/kg of the antibody to the male rabbit was shown to decrease in parallel the serum LH and testosterone concentrations by 80 to 90% of the normal level for a period of seven to ten days. The reproductive hormones returned to normal levels approximately two weeks after the initial injection. Similar observations were obtained with the female rabbits, in which the serum LH and estradiol concentrations were reversibly suppressed and recovered upon the same dose of single injection with the same antibody. These experiments support that GHR-106(hIgG4) can act on the anterior pituitary GnRH receptor as an antibody-based GnRH antagonist similar to Elagolix or Antide, except that GHR-106(hIgG4) has a much longer half-life (days vs. hours).

Besides proof-of-concept experiments in rabbits, quantitative RT-PCR experiments were performed and used as a tool to demonstrate almost a complete identity of the intracellular gene regulation between GHR-106 and decapeptide GnRH antagonists through in vitro studies. Therefore, it is reasonable to conclude that antibody-based and peptide-based GnRH antagonists are highly similar in terms of biological mechanisms of action, both against cancer cells and in the reversible suppressions of gonadotropin release by anterior pituitary, except that GHR-106 has a significantly longer half-life.

### Production of GHR-106-Related Antibody Drugs

GHR-106 of various isoforms including mouse-dog or mouse-cat chimeric forms can be mass-produced based on the knowledge and methods as are known in the art, including the US patents cited herein. For example, human (variable region) dog (constant Fc region) chimeric antibody, and mouse (variable region) dog (constant Fc Region) chimeric antibody can be mass produced based on the established knowledge for a GHR-106 antibody intended for administration to dogs.

Murine GHR-106 can be produced and purified through ascites fluid in mice or by in vitro culture methods of hybridoma cell lines. Humanized GHR-106 can be produced by permanent cell lines established previously. These include mGHR-106 (murine origin) GHR-106(hIgG1), and GHR-106(hIgG4) as well as different antibody fragments such as Fab, (Fab′)2 or single chain fragments of the variable region.

### Example 1: Proof-of-Concept Experiments in Rabbits and Implications for Widespread Clinical Applications in Humans and/or Domestic Animals

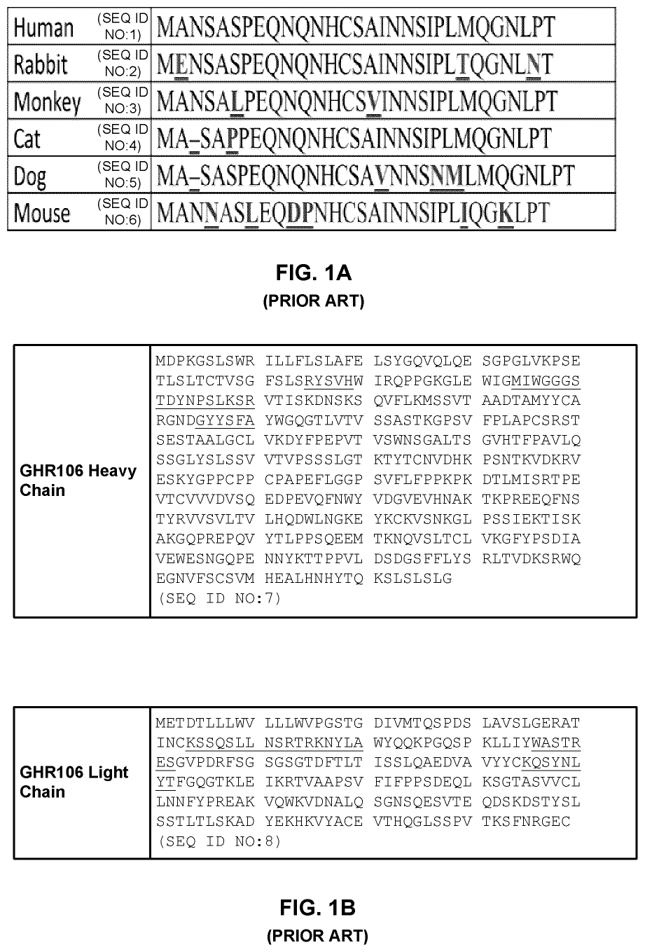

GHR-106 is a monoclonal antibody derived from the immunization of mouse against the N1-29 oligopeptide of human GnRH receptor. The amino acid sequences of the heavy chain and light chain of GHR-106 are shown in FIG. 1B, with the CDRs underlined. An animal model was selected to demonstrate that GHR-106 interacts with pituitary GnRH receptor which can lead to the reversible suppression of reproductive hormones in vivo. Therefore, N1-29 oligopeptides from different animal species including human, monkey, dog, cat, rabbit and mouse (SEQ ID NO: 1 to SEQ ID NO:6) are compared and shown in FIG. 1A for sequence homology. Based on the assumption that the highly similar sequence identities would lead to highly similar in vivo binding activities and comparable biological activities for their respective GnRH receptors, the rabbit was selected as a suitable animal model for proof-of-concept in vivo experiments in this study. Specifically, given the greater than 95% amino acid sequence similarity for the N1-N29 peptide of the GnRH receptors for humans and rabbits, the inventor predicts that their apparent K_{d}'s for binding to GHR-106 (hIgG4) are similar and so the rabbit was selected as a suitable animal to demonstrate reversible suppression of reproductive hormones such as LH, E2 and testosterone.

Based on the comparisons of N1-29 peptide sequences from different animal species, it can be concluded that GHR-106 may have a high degree of binding cross-reactivity between humans and several other animal species including rabbit, cat, dog and monkey. Therefore, GHR-106 can be potentially used as GnRH antagonist, not only for human applications, but also for those of several other animal species (mammals) including rabbit, dog and cat. The person skilled in the art can carry out established techniques (for example those used in the design of humanized antibodies) to design an antibody that is suitable for use in a different mammalian species to minimize the likelihood of undesirable cross-reactivity of the antibody.

### Example 2—Comparative ELISA Studies

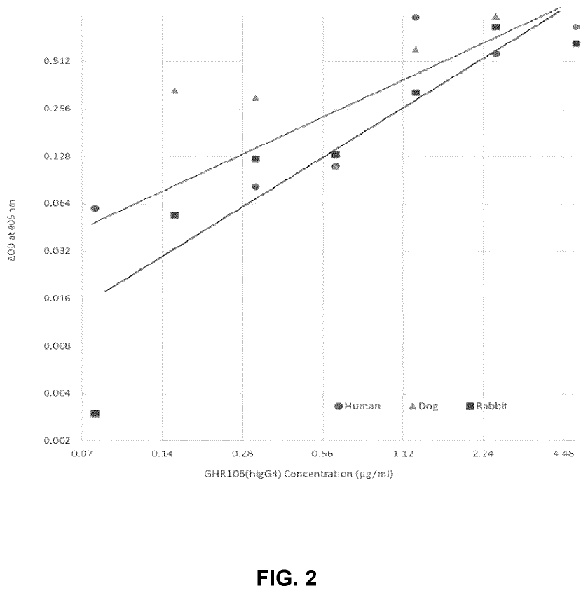

Binding studies between GHR-106 and N1-29 peptides derived from the animal species mentioned are essential to demonstrate comparable binding affinity of GHR-106 to N1-29 oligopeptides derived from human, dog, and rabbit, respectively. Therefore, comparative binding ELISA studies were performed to estimate the relative binding affinity between GHR-106 and microwell-coated N1-29 oligopeptides derived from human, dog and rabbit, respectively. The results of such binding studies are presented and compared in FIG. 2.

FIG. 2 shows a double log plot for ΔOD at 405 nm vs. GHR-106 antibody concentration where GHR-106 monoclonal antibody was applied to three separate well-coated N1-29 synthetic oligopeptides of GnRH receptor from humans, dog and rabbit, respectively. Unrelated RP215 monoclonal antibody was used as the negative control for any blank subtractions. Goat anti-human IgG labeled with alkaline phosphatase was used as the second antibody for signal detection. P-nitrophenyl phosphate was used as the substrate and monitored at 405 nm and double log plot presented. It was clearly demonstrated that the binding affinities between GHR-106 and N1-29 peptides derived from human, dog, and rabbit respectively are comparable to one another, when compared with that of unrelated RP215 as the negative control.

In previous studies, the inventor had showed that the three isoforms of GHR-106 including murine GHR-106, humanized GHR-106 and humanized GHR-106(hIgG4) are essentially identical in their respective binding affinity and specificity to human GnRH receptor as well as its N1-29 oligopeptide with dissociation constants on the order of 1-5 nM.

Based on ELISA binding studies presented in FIG. 2, it has been demonstrated that GHR-106 exhibits comparable binding to GnRH receptor or its N1-29 oligopeptides of either human or rabbit. Therefore, the rabbit was selected for proof-of-concept experiments to demonstrate the reversible suppression of reproductive hormones in vivo upon a single treatment with GHR-106.

### Example 3: Serum LH and Testosterone Concentrations Upon Injection of GHR-106 in Male Rabbits

Previous in vitro studies with human cancer cells have indicated that the apoptosis of cultured cancer cells can be induced after 24 to 72 hours following co-incubation with 1-10 μg/ml of GHR-106 monoclonal antibody in different isoforms. The degrees of induced apoptosis were comparable to those of the decapeptide GnRH antagonist, Antide, although the antibody is fifty times higher in molecular size.

To demonstrate that GHR-106 interacts with pituitary GnRH receptor similar to that of decapeptide GnRH antagonist, proof-of-concept experiments were performed in rabbits. In the case of male rabbits, serum concentrations of reproductive hormones including luteinizing hormone (LH) and testosterone were monitored regularly following a single subcutaneous injection with 3 mg/kg of hGHR-106.

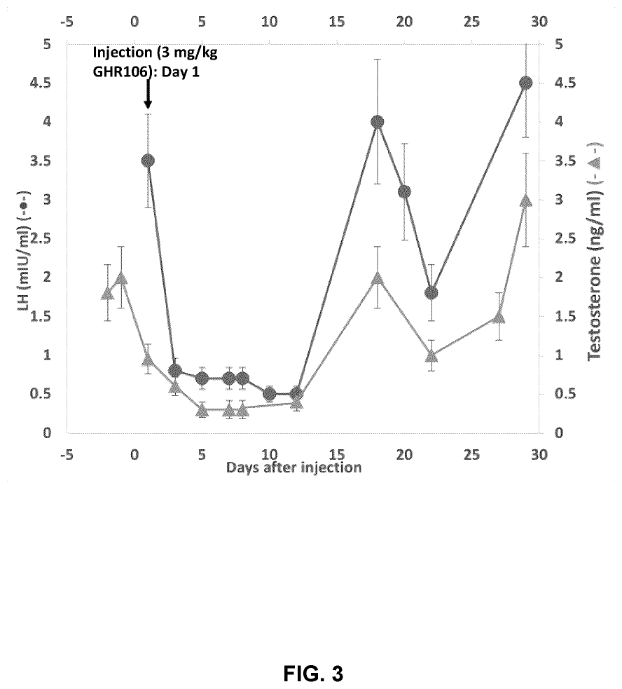

The serum LH and testosterone concentrations were determined, respectively, by EIA kits and plotted as a function of time during the day 1 to 30 time period. The results of the hormonal profiles are presented in FIG. 3, which shows serum LH (mIU/mL) and testosterone (ng/ml) concentrations plotted as a function of days after a single injection with 3 mg/kg of GHR-106 subcutaneously on day 1 to an adult male rabbit.

As shown in FIG. 3, upon a 3 mg/kg injection to a male rabbit, immediate suppressions of both serum LH and testosterone were observed after 24-48 hours (from 3.5 mlU/ml to <0.5 mlU/ml). The low LH levels were continued for at least one to two weeks. This was followed by a fluctuating increase in LH levels until the end of the third week to reach stable normal range (2.9-5.0 mlU/ml). Without being bound by theory, it is noted that fluctuations in the levels of reproductive hormones in mammals are commonly observed on a day-to-day basis in mammals, for example due to endocrinological, environmental or physiological reasons, and so some fluctuation in the level of reproductive hormone is expected. However, the trend of the reversible suppression of reproductive hormones following the administration of GHR-106 is readily observable and consistent in these examples.

Similarly, upon a single injection of 3 mg/kg dose to a male rabbit, the serum testosterone concentrations decreased by more than 80% from 0.95 ng/ml to ≤0.1 ng/ml during the first two weeks. The time-dependent serum levels of testosterone are synchronised with those of LH. During the third week after the injections, the fluctuating changes of LH and testosterone levels are parallel to each other until day 30 when the hormone levels are within the normal range (FIG. 3).

### Example 4: Serum LH and Estradiol Concentrations Upon a Single Injection of hGHR-106 in Female Rabbits

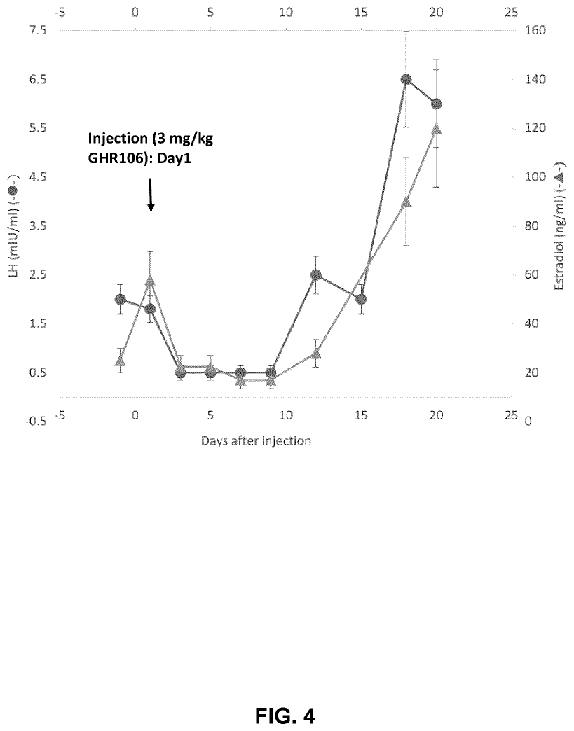

The hormonal profiles of serum luteinizing hormone (LH) and Estradiol (E2) in female rabbits were monitored upon a single injection with a 3 mg/kg dose of hGHR-106. The serum concentrations of LH and E2 were also determined regularly from Day 1 to Day 20 and presented in FIG. 4.

The suppression of LH levels was observed immediately during the first few days after the antibody injection (from 3 mlU/ml to ≤1 mlU/ml). Similarly, the serum E2 concentrations were decreased in parallel during the same time period (from 50 pg/ml to ≤20 pg/ml) until day 10.

From day 10 to day 20, both LH and E2 concentrations increased with time and reached 6 mIU/ml and 120 pg/ml, respectively after day 18 and 20.

At a lower dose injection of 1 mg/kg to separated females, the overall profiles of LH and E2 concentrations were similar to those of the high dose during the same observation period (data not presented).

### Example 5—Quantitative Gene Regulation Studies

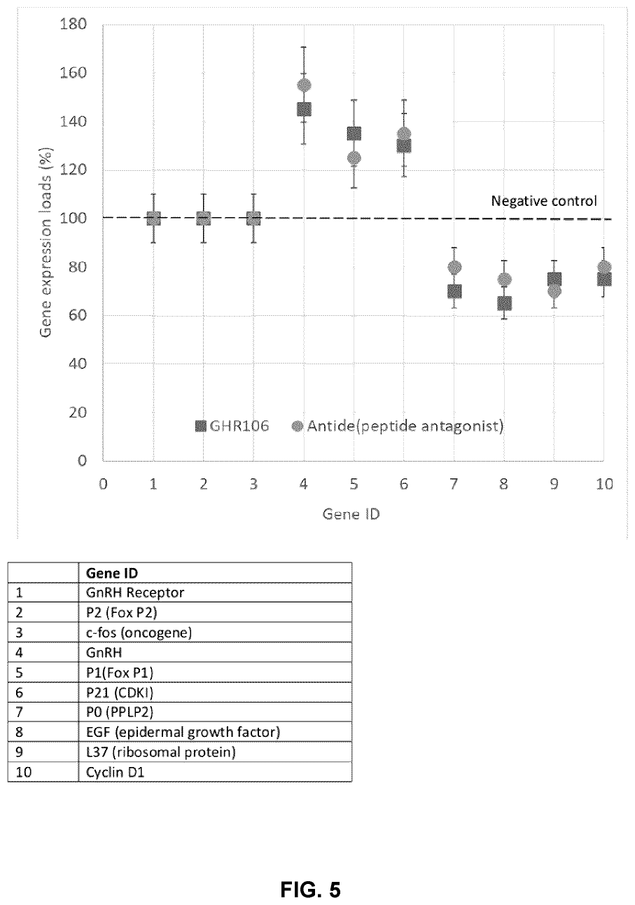

Quantitative Gene Regulation Studies were performed to justify a complete identity of molecular mechanisms between GHR-106 and the peptide GnRH antagonist antide. Quantitative changes of gene expression upon binding to human GnRH receptor were presented by using either GHR-106 or the peptide GnRH antagonist antide after incubation with human cancer cells. To make further comparisons between GHR-106 and decapeptide GnRH antagonists, ten regulatory genes were selected for quantitation by RTPCR methods and results are presented and compared in FIG. 5. The results of these comparative studies revealed strong similarity in terms of molecular mechanisms of action between GHR-106 and Antide to cancer cells.

### Example 6—High Specificity of GHR-106 Monoclonal Antibody

Testing reveals the high specificity of GHR-106 to human GnRH receptor as compared to many other known and available anti GnRH receptor monoclonal antibodies. In particular, the ability of GHR-106 to detect overexpression of GnRHR in a reference cell line is tested and compared with four different commercially available antibodies. It is found that only the GHR-106 antibody is able to detect overexpression of GnRHR in the reference cell line.

Given this tissue-specificity in humans that is not observed in other antibodies, GHR-106 may be one of the best antibodies generated to react with human GnRH receptor, respectively, as well as the N1-29 oligopeptide of this receptor.

In addition, the other examples described herein also reveal a high degree of species cross-reactivity among several different animal species. Therefore, GHR-106 should be recognized as a third class of therapeutic, i.e. an antibody-based GnRH antagonist which is comparable to organic chemical or decapeptide-based GnRH antagonists.

Humanized forms of GHR-106 can only be utilized for human clinical application either in cancer therapy or in fertility control, due to the intrinsic and restricted immunogenicity upon applications in humans. This concern may be considered so suitable modifications to the antibody, and in particular the Fc regions of the antibody, can be made for clinical applications in other animal species, including other mammalian species. For example, in order to avoid allergic reactions to allogeneic injections, pure-bred mouse-derived antibodies can be replaced with chimeric antibodies from another species, e.g. dogs or cats (a receptor-constant region). To minimize heterologous immune response, mouse (variable, VR)-dog (constant, Fc) chimeric IgG can be produced and used for applications in dogs. Similarly, mouse-cat chimeric antibody can be generated according to known methods for application in cats. Similar modifications can be made for application of the GHR-106 antibody as a therapeutic agent in other species.

### Example 7—Proof of Concept Rabbit Experiments in Large Scale

To demonstrate that GHR-106(hIgG4) acts as a GnRH antagonist in vivo, large scale proof of concept experiments were performed in rabbits and data presented in these additional examples.

Judging from the data from the above examples, reversible suppression of reproductive (i.e. sex related) hormones including luteinizing hormone, testosterone and estradiol was observed upon single injections of male or female rabbits. The serum levels of reproductive hormones returned to normal ranges following one to two weeks after injection.

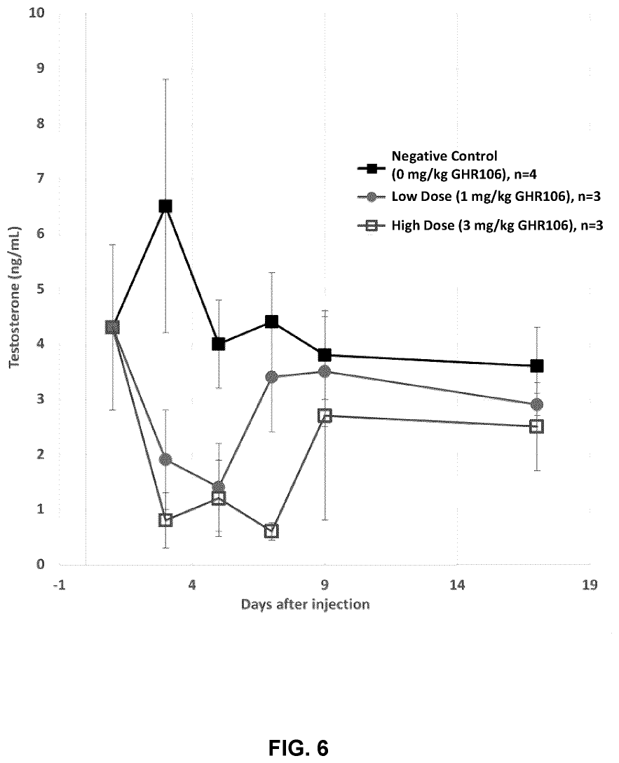

With preliminary observations in single rabbit data, large scale rabbit experiments (n≥30) including negative controls were conducted with identical protocols. Specifically, to further confirm that GHR-106 acts as a GnRH antagonist, large scale experiments were performed in male and female rabbits. The data generated in each experimental group were analyzed statistically. The means and standard deviations of hormone levels from rabbits in each experimental group are presented in FIGS. 6-9 and corresponding selected statistical analyses and separate comparisons with those of the negative controls are presented in Table 1 to Table 4.

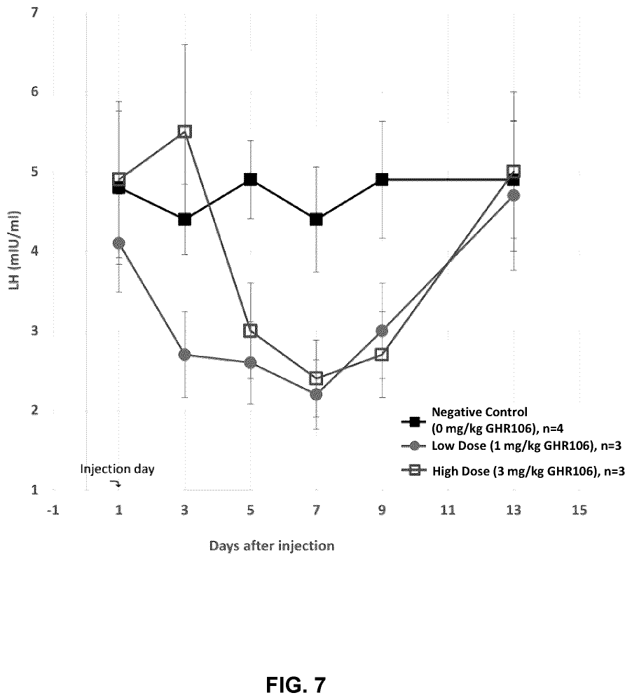

In the case of male rabbits, the serum concentrations of reproductive hormones including LH and testosterone of ten male rabbits were monitored and measured after a single subcutaneous injection with 1 mg/kg of GHR-106 (low dosage, n=3) or 3 mg/kg of GHR-106 (high dosage, n=3) on day 1, or after no injection (negative control, n=4). Serum testosterone levels were determined and the averages (with standard deviations) were determined from day 1 to day 17, and serum LH levels were determined starting on day 1 and ending on day 13 (with standard deviations) The results of the testosterone profile are found in FIG. 6. The results of the LH profile are found in FIG. 7. Selected statistical analyses and separate comparisons with those of the negative controls for FIG. 6 and FIG. 7 are found in Table 1 and Table 2, respectively.

As seen in FIG. 6 and FIG. 7, the serum testosterone and LH levels of the male rabbits were reversibly suppressed by a single injection of GHR-106 on day 1 at either the low dosage or the high dosage. Treatment with either the low dosage or the high dosage resulted in an immediate and statistically significant decrease in serum testosterone and LH compared to the negative control. Notably, a similar magnitude of suppression was observed in both the low dosage and high dosage groups. The serum testosterone and LH of the rabbits in the low dosage and high dosage groups subsequently returned to levels similar to those in the negative control group after several days of suppression.

TABLE 1
Selected statistical analyses of testosterone
profiles in male rabbits in FIG. 6
t-test values and p-values

| | |
|---|---|
| neg. ctrl to 1 mg/kg | neg. ctrl to 3 mg/kg |

| Day | t test | P value | t test | P value |
|---|---|---|---|---|
| 3 | 2.965789 | 0.003072 | 4.2 | 0.0001 |
| 5 | 3.352632 | 0.000842 | 3.851228 | 0.000132 |
| 7 | 1.085873 | 0.281089 | 7.396226 | 0.0001 |
| 9 | 0.34386 | 0.743985 | 0.840546 | 0.407961 |

TABLE 2
Selected statistical analyses of LH profiles in male rabbits in FIG. 7
t-test values and p-values

| | |
|---|---|
| neg. ctrl to 1 mg/kg | neg. ctrl to 3 mg/kg |

| Day | t test | P value | t test | P value |
|---|---|---|---|---|
| 3 | 3.578947 | 0.000373 | 1.473684 | 0.140847 |
| 5 | 4.69828 | 0.0001 | 3.59633 | 0.00035 |
| 7 | 4.126316 | 0.0001 | 3.619575 | 0.000321 |
| 9 | 2.93633 | 0.003372 | 3.559959 | 0.0004 |

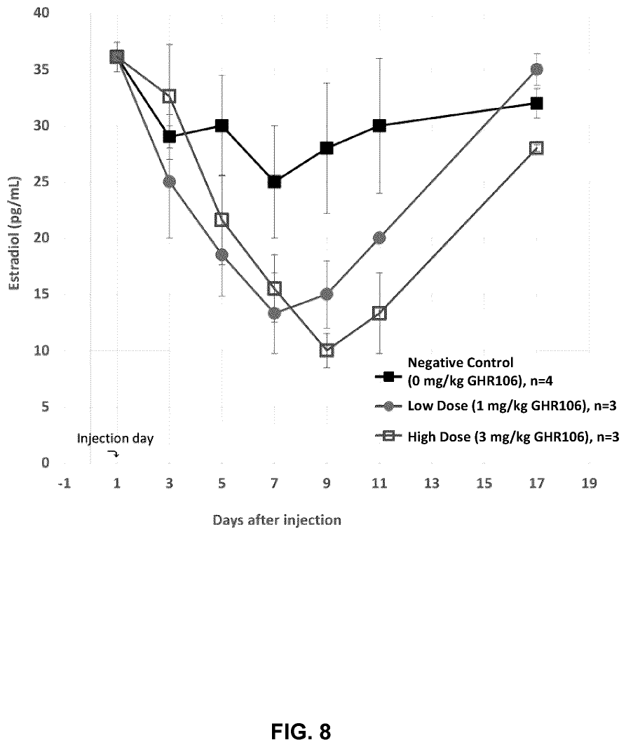

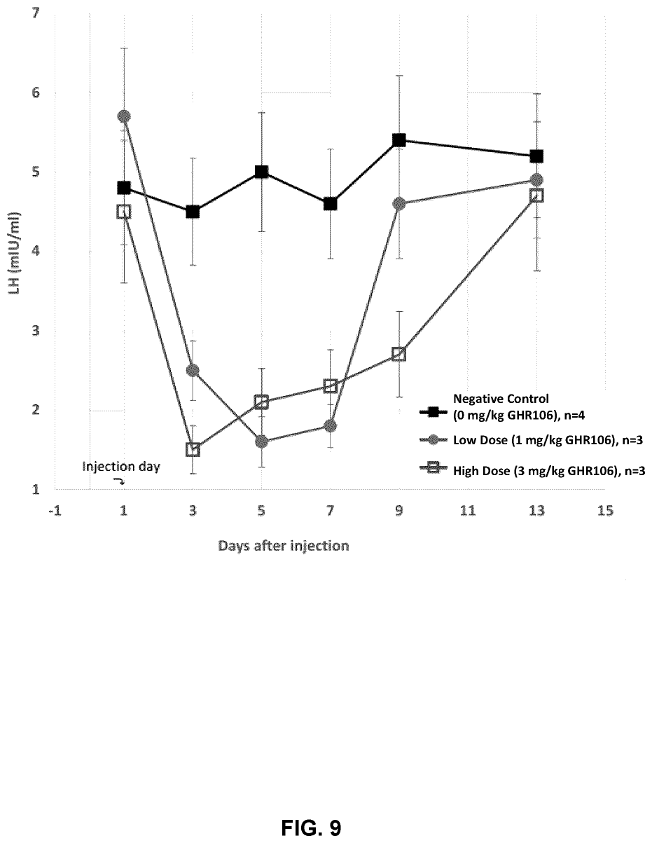

In the case of female rabbits, the serum concentrations of reproductive hormones including LH and estradiol (E2) of ten female rabbits were monitored and measured after a single subcutaneous injection with 1 mg/kg of GHR-106 (low dosage, n=3) or 3 mg/kg of GHR-106 (high dosage, n=3) on day 1, or after no injection (negative control, n=4). Serum E2 levels were determined starting on day 1 and ending on day 17, and serum LH levels were determined starting on day 1 and ending on day 13. The results of the estradiol (E2) profile are found in FIG. 8. The results of the LH profile are found in FIG. 9. Selected statistical analyses and separate comparisons with those of the negative controls for FIG. 8 and FIG. 9 are found in Table 3 and Table 4, respectively.

As seen in FIG. 8 and FIG. 9, the serum estradiol (E2) and LH levels of the female rabbits were reversibly suppressed by a single injection of GHR-106 on day 1 at either the low dosage or the high dosage. Treatment with either the low dosage or the high dosage resulted in an immediate and statistically significant decrease in serum estradiol (E2) and LH compared to the negative control. Notably, a similar magnitude of suppression was observed in both the low dosage and the high dosage groups. The serum estradiol (E2) and LH of the rabbits in the low dosage and the high dosage groups subsequently returned to levels similar to those in the negative control group after several days of suppression.

TABLE 3
Selected statistical analyses of estradiol
(E2) profiles in female rabbits in FIG. 8
t-test values and p-values

| | |
|---|---|
| neg. ctrl to 1 mg/kg | neg. ctrl to 3 mg/kg |

| Day | t test | P value | t test | P value |
|---|---|---|---|---|
| 3 | 1.178947 | 0.240867 | 1.125359 | 0.263496 |
| 5 | 2.893453 | 0.003859 | 2.038885 | 0.041122 |
| 7 | 2.806854 | 0.005042 | 2.45 | 0.014212 |
| 9 | 3.047847 | 0.002359 | 5.087239 | 0.0001 |

TABLE 4
Selected statistical analyses of LH profiles in female rabbits in FIG. 9
t-test values and p-values

| | |
|---|---|
| neg. ctrl to 1 mg/kg | neg. ctrl to 3 mg/kg |

| Day | t test | P value | t test | P value |
|---|---|---|---|---|
| 3 | 3.929825 | 0.0001 | 6.348178 | 0.0001 |
| 5 | 6.555829 | 0.0001 | 5.11381 | 0.0001 |
| 7 | 6.017544 | 0.0001 | 4.126316 | 0.0001 |
| 9 | 1.100351 | 0.274547 | 4.126316 | 0.0001 |

In general, both single or multi-rabbit experiments revealed reversible suppression of reproductive hormones upon a single injection of GHR-106(hIgG4) with either 1 mg/kg or 3 mg/kg dose. The hormone levels return to normal ranges as in the negative control group after 1-3 weeks, indicating the suppression of the sex related hormones is reversible. Thus, consistent with the individual rabbit data described above, effects of reversible hormone suppression were observed among the larger experimental group.

In conclusion, multi-rabbit and individual rabbit experiments led to the same conclusion in terms of time-dependent reversible sex related hormone suppression upon GHR-106 antibody treatments. Therefore, the inventor has demonstrated that GHR-106(hIgG4) is an antibody-based long-acting GnRH antagonist, which exhibits biological effects comparable with the decapeptide GnRH antagonist such as cetrorelix, currently in use clinically, but with potential benefits of a longer period of activity given its longer half life.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

Without limiting the foregoing, various embodiments include a number of aspects including the following. These aspects are based on the examples disclosed in this application, which demonstrate factors including (1) a high degree of amino acid sequence homology and broad degrees of species-cross reactivity of GHR-106 to several animal species; (2) proof-of-concept experiments providing direct evidence of strong interactions between GHR-106 and GnRH receptor in humans or other mammalian species; and (3) a high degree of identity and consistency in quantitative gene expression level changes between the long acting antibody-based and short-lived peptide-based GnRH antagonists.

In a first aspect, GHR-106 of various isoforms or species cross-reacts with GnRH receptors of several different animal species (dog, cat and rabbit and monkey) and can be used as a GnRH antagonist, as long as they share with humans a high degree of sequence homology in N1-29 oligopeptide of their respective receptors (≥90-95%).

In a second aspect, as a GnRH antagonist, GHR-106 can be used to reversibly suppress the endogeneous reproductive hormones (eg: LH, FSH, testosterone, estradiol and progesterone etc.) of humans or any other animal species which meet the criteria under the first aspect.

In a third aspect, as a GnRH antagonist, GHR-106 in humanized IgG4 isotype [GHR-106(hIgG4)] can act directly on human anterior pituitary for reversible suppression of reproductive hormones upon GHR-106 treatment to manipulate GnRH receptor-controlled fertility regulations or disorders, similar to the drug actions of decapeptide analogs such as cetrorelix.

In a fourth aspect, as a GnRH antagonist, GHR-106 in various isoforms or species can be used in cancer therapy of almost all receptor-positive cancer not only in humans, but also several other animal species, including dog, cat and rabbit, etc.

1. A method of using a GHR-106 monoclonal antibody or an antigen-binding fragment thereof to regulate a level of a sex related hormone in a mammalian subject, the method comprising administering the GHR-106 monoclonal antibody or an antigen-binding fragment thereof to the mammalian subject.

2. The method as defined in claim 1, wherein the GHR-106 monoclonal antibody or the antigen-binding fragment thereof causes a reversible suppression of at least one sex related hormone in the subject.

3. The method as defined in claim 2, wherein the reversible suppression of the at least one sex related hormone comprises a decrease in serum levels of the at least one sex related hormone in the subject for between 3 days and 21 days after administration of the GHR-106 monoclonal antibody or the antigen-binding fragment thereof.

4. The method as defined in claim 1, wherein the at least one sex-related hormone is testosterone, estradiol, luteinizing hormone, progesterone, follicle stimulating hormone, or a combination thereof.

5. The method as defined in claim 1, to terminate an ectopic pregnancy.

6. The method as defined in claim 1, wherein the GHR-106 monoclonal antibody or antigen-binding fragment thereof is used to control ovulation in a female subject.

7. The method as defined in claim 1, wherein the GHR-106 monoclonal antibody or antigen-binding fragment thereof is used for fertility control in a female subject.

8. The method as defined in claim 1, wherein the GHR-106 monoclonal antibody or antigen-binding fragment thereof is used for fertility control in a male subject.

9. A method of treating a sex hormone-related condition or disorder in a mammalian subject comprising carrying out the method as defined in claim 1, optionally wherein the sex hormone-related condition or disorder is a reproductive disease, medical transition for transgender people, infertility, assisted reproductive therapy, contraception, endometriosis, endometrial thinning, adenomyosis, endometrial hyperplasia, uterine leiomyoma, premenstrual syndrome, benign prostatic hypertrophy, ovarian disorders, polycystic ovary disease, or precocious puberty; optionally wherein the sex hormone-related condition or disorder is a condition that is known to be treatable by the administration of known GnRH antagonists, wherein the known GnRH antagonists optionally comprise antide or cetrorelix.

10. (canceled)

11. (canceled)

12. The method as defined in claim 9, wherein the sex hormone-related condition or disorder is one in which a longer half-life of an active treatment agent in circulation than known decapeptide GnRH antagonists is desirable.

13. The method as defined in claim 1, wherein the GHR-106 monoclonal antibody or the antigen-binding fragment thereof is administered at a dosage of between about 1 mg/kg to about 3 mg/kg relative to the weight of the subject; or wherein the GHR-106 monoclonal antibody or antigen-binding fragment thereof is administered at a dose of between about 50 mg to about 300 mg; or wherein the GHR-106 monoclonal antibody or antigen-binding fragment thereof is administered at repeated spaced apart intervals of between about every 1 weeks to about every 3 weeks.

14. (canceled)

15. (canceled)

16. The method as defined in claim 1, wherein the GHR-106 monoclonal antibody or the antigen-binding fragment thereof has a heavy chain having an amino acid sequence that has at least 90% sequence identity to the amino acid sequence of SEQ ID NO:7; and/or wherein the GHR-106 antibody has a light chain having an amino acid sequence that has at least 90% sequence identity to the amino acid sequence of SEQ ID NO:8.

17. The method as defined in claim 1, wherein:
a) the CDR1 region of the heavy chain of the GHR-106 monoclonal antibody or the antigen-binding fragment thereof has the amino acid sequence RYSVH (SEQ ID NO:9); and
b) the CDR2 region of the heavy chain of the GHR-106 monoclonal antibody or the antigen-binding fragment thereof has the amino acid sequence MIWGGGSTDYNPSLKSR (SEQ ID NO:10); and
c) the CDR3 region of the heavy chain of the GHR-106 monoclonal antibody or the antigen-binding fragment thereof has the amino acid sequence GYYSFA (SEQ ID NO:11); and
d) the CDR1 region of the light chain of the GHR-106 monoclonal antibody or the antigen-binding fragment thereof has the amino acid sequence KSSQSLLNSRTRKNYLA (SEQ ID NO:12); and
e) the CDR2 region of the light chain of the GHR-106 monoclonal antibody or the antigen-binding fragment thereof has the amino acid sequence WASTRES (SEQ ID NO:13); and
f) the CDR3 region of the light chain of the GHR-106 monoclonal antibody or the antigen-binding fragment thereof has the amino acid sequence KQSYNLYT (SEQ ID NO:14).

18. (canceled)

19. The method as defined in claim 1, wherein the GHR-106 antibody or the antigen-binding fragment thereof acts similarly to known decapeptide GnRH antagonists, optionally wherein the known decapeptide GnRH antagonists are antide or cetrorelix.

20. (canceled)

21. The method as defined in claim 1, wherein the subject is male.

22. The method as defined in claim 1, wherein the subject is female.

23. The method as defined in claim 1, wherein the antigen-binding fragment of the GHR-106 monoclonal antibody comprises an IgG antibody fragment, wherein the IgG antibody fragment optionally comprises an F(ab′)2, Fab, scFab or scFv.

24. The method as defined in claim 1, wherein the subject is a human, and wherein the GHR-106 monoclonal antibody or the antigen-binding fragment thereof comprises a humanized GHR-106 monoclonal antibody or antigen-binding fragment thereof.

25. The method as defined in claim 1, wherein the subject is a monkey, rabbit, cat or dog.

26. The method as defined in claim 1, wherein the subject is a mammal in which the N1-29 amino acid sequence of the GnRH receptor has at least 90% sequence identity to SEQ ID NO:1.

27. The method as defined in claim 26, wherein the GHR-106 monoclonal antibody or antigen-binding fragment thereof is a chimeric antibody engineered to contain the Fc regions of IgG4 in the subject's species.

28. The method as defined in claim 1, wherein the GHR-106 monoclonal antibody or the antigen-binding fragment thereof has a half-life in human circulation of between 3 days to 21 days.


SEQUENCE LISTING (14 sequences)

SEQ ID NO: 1 - protein, 30 aa, Homo sapiens
MANSASPEQNQNHCSAINNSIPLMQGNLPT

SEQ ID NO: 2 - protein, 30 aa, Oryctolagus cuniculus
MENSASPEQNQNHCSAINNSIPLTQGNLNT

SEQ ID NO: 3 - protein, 30 aa, Pan troglodytes
MANSALPEQNQNHCSVINNSIPLMQGNLPT

SEQ ID NO: 4 - protein, 29 aa, Felis catus
MASAPPEQNQNHCSAINNSIPLMQGNLPT

SEQ ID NO: 5 - protein, 29 aa, Canis familiaris
MASASPEQNQNHCSAVNNSNMLMQGNLPT

SEQ ID NO: 6 - protein, 30 aa, Mus musculus
MANNASLEQDPNHCSAINNSIPLIQGKLPT

SEQ ID NO: 7 - protein, 468 aa, Artificial Sequence
  Artificial Construct
MDPKGSLSWRILLFLSLAFELSYGQVQLQESGPGLVKPSETLSLTCTVSGFSLSRYSVHWIRQPPGKGLEWIGMIWGGGSTDYNPSLKSRVTISKDNSKSQVFLKMSSVTAADTAMYYCARGNDGYYSFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 8 - protein, 239 aa, Artificial Sequence
  Artificial Construct
METDTLLLWVLLLWVPGSTGDIVMTQSPDSLAVSLGERATINCKSSQSLLNSRTRKNYLAWYQQKPGQSPKLLIYWASTRESGVPDRFSGSGSGTDFTLTISSLQAEDVAVYYCKQSYNLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 5 aa, Artificial Sequence
  Artificial Construct
RYSVH

SEQ ID NO: 10 - protein, 17 aa, Artificial Sequence
  Artificial Construct
MIWGGGSTDYNPSLKSR

SEQ ID NO: 11 - protein, 6 aa, Artificial Sequence
  Artificial Construct
GYYSFA

SEQ ID NO: 12 - protein, 17 aa, Artificial Sequence
  Artificial Construct
KSSQSLLNSRTRKNYLA

SEQ ID NO: 13 - protein, 7 aa, Artificial Sequence
  Artificial Construct
WASTRES

SEQ ID NO: 14 - protein, 8 aa, Artificial Sequence
  Artificial Construct
KQSYNLYT